(19) United States
(12) Patent Application Publication
(10) Pub. No.: US 2006/0287269 A1
(43) Pub. Date: Dec. 21, 2006

(54) Short interfering nucleic acid hybrids and methods thereof

(75) Inventors: Allen T. Christian, Madison, WI (US); Janelle S. Lamberton, Livermore, CA (US)

Correspondence Address: Assistant Laboratory Counsel;Lawrence Livermore National Laboratory, P.O. Box 808, L-703, Livermore, CA, 94551, US

(73) Assignee: The Regents of the University of California

(21) Appl. No.: 11/434,836

(22) Filed: May 15, 2006

Related U.S. Application Data
(63) Continuation-in-part of application No. 10/410,220, filed on Apr. 8, 2003, now abandoned.
(60) Provisional application No. 60/409,680, filed on Sep. 9, 2002.

(51) Int. Cl. A61K 48/00; C07H 21/02

(52) U.S. Cl. 514/44; 536/23.1

(57) ABSTRACT

Disclosed herein are siHybrids used for gene silencing. An siHybrid is a short double-stranded molecule comprised of one strand of DNA and one strand of RNA, annealed together, with a 2-base overhang at each 3′ end. In addition to DNA and RNA, it may contain PNA or other nucleic acid analogs. siHybrids can silence a gene with greater magnitude and duration than siRNA. sihybrids are ideal candidates for pharmaceutical and therapeutic agents for treating diseases caused by an over-expressed gene or a cancerous gene. siHybrids can be used as antivirus agents, fungicides, herbicides or pesticides. An appropriate siHybrid can be designed to silence any gene in any eukaryotic cell or organism.

### CROSS-REFERENCE TO RELATED APPLICATIONS

This application is a Continuation-in-Part of co-pending U.S. patent application Ser. No. 10/410,220 filed Apr. 8, 2003 and titled, “Short-Interfering Nucleic Acid Hybrids and Methods Thereof,” which claims the benefit of U.S. Provisional Patent Application No. 60/409,680 filed Sep. 9, 2002 and titled “Gene Silencing Using DNA-RNA-Short, Interfering Molecules”.

The United States Government has rights in this invention pursuant to Contract No. W-7405-ENG-48 between the United States Department of Energy and the University of California for the operation of Lawrence Livermore National Laboratory.

### BACKGROUND

In recent years it has been accepted that RNA interference is mediated by short interfering RNA molecules (“siRNA”) that exhibit sequence specific gene silencing effects. Although the detailed mechanism of siRNA gene silencing is not fully understood, genes can be silenced or disabled by degradation of cellular mRNA by introducing an siRNA molecule that is homologous to the target genes.

Previous experimental work involving the use of antisense molecules demonstrated antisense therapy as an excellent antiviral infectant, but its utility was offset by the fact that the half-life of antisense molecules is very short. Also, antisense therapy is a passive process in that it simply blocks the translation of the viral mRNA, whereas RNAi actually degrades the mRNA. Similar work involving the transfection of an siRNA-producing plasmid into cells works well for mutagenesis studies, but an active process such as this may not be as useful for long-term protection from a genetic process, such as microbial infection.

The following references are related to gene silencing technology and are hereby incorporated by reference in their entirety.

### REFERENCES

1. Fire, A., Xu, S., Montgomery, M. K., Kostas, S. A., Driver, S. E., and Mello, C. C. (1998) Potent and specific genetic interference by double stranded RNA in Caenorhabditis elegans. Nature. 408, 325-330.
2. Kennerdell, J. R., and Carthew, R. W. (1998) Use of dsRNA-mediated genetic interference to demonstrate that frizzled and frizzled 2 act in the wingless pathway. Cell. 95, 1017-1026.
3. Misquitta, L., and Paterson, B. M. (1999) Targeted disruption of gene function in Drosophila by RNA interference (RNA-i): a role for nautilus in embryonic somatic muscle formation. Proc. Natl. Acad. Sci. USA. 96, 1451-1456.
4. Hammond, S. M., Bernstein, E., Beach, D., and Hannon, G. J. (2000) An RNA-directed nuclease mediates post transcriptional gene silencing in Drosophila cells. Nature. 404, 293-296.
5. Lohmann, J. U., Endl, I., and Bosch, T. C. (1999) Silencing of developmental genes in Hydra. Dev. Biol. 214, 211-214.
6. Wargelius, A., Ellingsen, S., and Fjose, A. (1999) Double stranded RNA induces specific developmental defects in zebrafish embyos. Biochem. Biophys. Res. Commun. 263, 156-161.
7. Ngo, H., Tschudi, C., Gull, K., and Ullu, E. (1998) Double stranded RNA induces mRNA degradation in Trypanosoma brucei. Proc. Natl. Acad Sci. USA. 95, 14687-14692.
8. Montgomery, M. K., Xu, S., Fire, A. (1998) RNA as a target of double stranded RNA mediated genetic interference in Caenorhabiditis elegans. Proc. Natl. Acad. Sci. USA. 95, 15502-15507.
9. Bosher, J. M., Dufourcq, P., Sookhareea, S., Labouesse, M. (1999) RNA interference can target pre-mRNA. Consequences for gene expression in Caenorhabiditis elegans operon. Genetics. 153, 1245-1256.
10. Fire, A. (1999) RNA-triggered gene silencing. Trends Genet. 15, 358-363.
11. Sharp, P. A. (1999) RNAi and double-stranded RNA. Genes Dev. 13, 139-141.
12. Ketting, R. F., Harerkamp, T. H., van Luenen, H. G., and Plasterk, R. H. (1999) Mut-7 of C. elegans, required for transposon silencing and RNA interference, is a homolog of Werner syndrome helicase and RNase I. Cell. 99, 133-141.
13. Tabara, H., Sarkissian, M., Kelly, W. G., Fleenor, J., Grishok, A., Timmons, L., Fire, A., and Mello, C. C. (1999) The rde-1 gene, RNA interference, and transposon silencing in C.elegans. Cell. 99, 123-132.
14. Zamore, P. D., Tuschl, T., Sharp, P. A., and Bartel, D. P. (2000) RNAi: Double stranded RNA directs the ATP dependent cleavage of mRNA at 21 to 23 nucleotide intervals. Cell. 101, 25-33.
15. Bernstein, E., Caudy, A. A., Hammond, S. M., and Hannon, G. J. (2001) Role for a bidentate ribonuclease in the initiation step of RNA interference. Nature. 409, 363-366.
16. Elbashir, S., Lendeckel, W., and Tuschl, T. (2001) RNA interference is mediated by 21 and 22 nucleotide RNAs. Genes and Dev. 15, 188-200.
17. Sharp, P. A. (2001) RNA interference 2001. Genes and Dev. 15, 485-490.
18. Hunter, T., Hunt, T., and Jackson, R. J. (1975) The characteristics of inhibition of protein synthesis by double-stranded ribonucleic acid in reticulocyte lysates. J. Biol. Chem. 250, 409-417.
19. Bass, B. L. (2001) The short answer. Nature. 411, 428-429.
20. Elbashir, S. M., Harborth, J., Lendeckel, W., Yalcin, A., Weber, K., and Tuschl, T. (2001) Duplexes of 21-nucleotide RNAs mediate RNA interference in cultured mammalian cells. Nature. 411, 494-498.
21. Carson, P. E. and Frischer, H. (1966) Glucose-6-Phosphate dehydrogenase deficiency and related disorders of the pentose phosphate pathway. Am J Med. 41, 744-764.
22. Stamato, T. D., Mackenzie, L., Pagani, J. M., and Weinstein, R. (1982) Mutagen treatment of single Chinese Hamster Ovary cells produce colonies mosaic for Glucose-6-phosphate dehydrogenase activity. Somatic Cell Genetics. 8, 643-651.

### SUMMARY OF THE INVENTION

The present invention provides a novel composition and method of using the composition to inhibit gene function in any eukaryotic organism or cell, both in vivo and in vitro. The short interfering nucleic acid or nucleic acid analog hybrids of this invention may be used to target and inhibit the function of any gene for which a specific sequence can be identified regardless of the function or the source of the gene.

In specific embodiments, the present invention provides a composition that is composed of hybridized complementary portions of single strands of nucleic acids or nucleic acid analogs that are hybridized to other single strands of different types of nucleic acids or nucleic acid analogs to form an siHybrid that has a hybridized portion and at least one 3′ overhang. The hybridized portion of the siHybrid may be as long as from ten to one hundred base pairs in length, depending on the gene and the organism or cell to which it is to be applied.

The present invention also provides a composition that is composed of hybridized complementary portions of single strands of nucleic acids or nucleic acid analogs that are hybridized to other single strands of different types of nucleic acids or nucleic acid analogs to form an siHybrid that has a hybridized portion that has a length of 19 to 21 base pairs and two 3′ overhangs that are 2-3 bases in length.

Additionally, the present invention provides a composition that is composed of hybridized complementary portions of single strands of nucleic acids or nucleic acid analogs that are hybridized to other single strands of different types of nucleic acids or nucleic acid analogs to form an siHybrid that has a hybridized portion that has a length of 21 base pairs and two 3′ overhangs that are 2 bases in length.

The invention also provides a method for making the siHybrid compositions by providing single strands of nucleic acids or nucleic acid analogs that are hybridized to other single strands of different types of nucleic acids or nucleic acid analogs to form an siHybrid that has a hybridized portion and at least one 3′ overhang.

The invention furthermore provides a method for making the siHybrid compositions by providing single strands of nucleic acids or nucleic acid analogs that are hybridized to other single strands of different types of nucleic acids or nucleic acid analogs to form an siHybrid that has a hybridized portion that has a length of 19 to 21 base pairs and two 3′ overhangs that are 2-3 bases in length.

Additionally, the invention provides a method for making the siHybrid compositions by providing single strands of nucleic acids or nucleic acid analogs that are hybridized to other single strands of different types of nucleic acids or nucleic acid analogs to form an siHybrid that has a hybridized portion that has a length of 21 base pairs and two 3′ overhangs that are 2 bases in length.

The invention also provides a method for making a plurality of siHybrid compositions by providing multiple single strands of nucleic acids or nucleic acid analogs that are hybridized to other multiple single strands of different types of nucleic acids or nucleic acid analogs to form a plurality of sihybrids that have hybridized portions that have a length of 19 to 21 base pairs and at least one 3′ overhang that is 2 to 3 bases in length.

The invention also provides a method for making a plurality of siHybrid compositions by providing multiple single strands of nucleic acids or nucleic acid analogs that are hybridized to other multiple single strands of different types of nucleic acids or nucleic acid analogs to form a plurality of siHybrids that have hybridized portions that have a length of 21 base pairs and two 3′ overhangs that are 2 bases in length.

A further embodiment of the invention is a method of applying the siHybrids directly to a substrate or to a substrate using a transfecting agent to silence a single gene or a plurality of genes, where the substrate is a eukaryotic cell or organism.

### BRIEF DESCRIPTION OF THE DRAWINGS

FIG. 1 illustrates an example of an siHybrid molecule; in this example, the top strand (SEQ ID NO:2) is DNA and the bottom strand (SEQ ID NO:1) is RNA; the complementary portion is boxed and labeled “2”; the overhangs are boxed and labeled “4”.

FIG. 2 is a bar graph illustrating the effects of siRNA and siHybrid treatment on G6PD expression in CHO cells as detected using a G6PD colorimetric assay based on a tetrazolium-based histochemical stain containing G6P and NADP and quantification of degree of color of cells, as described in Materials and Methods. Expression level obtained using the positive control was defined as 100%. Positive control: non-homologous sequence (T7 primer); siRNA and siHybrid: 21 base sequence from hamster G6PD gene. For this experiment, delivery of siRNA and siHybrid was unaided by transfection media or agents.

FIG. 3 illustrates the effects on G6PD expression in CHO cells of siRNA (FIG. 3A), siDNA (FIG. 3B), DNAs:RNAa sihybrid (FIG. 3C) and RNAs:DNAa sihybrid (FIG. 3D). Expression was assayed as in FIG. 2. Expression level obtained using the positive control was defined as 100%. Key: “+”: positive control (no transfection); “−”: negative control (cells incubated with stain not containing G6P); B: blank (transfection without nucleic acid); N: non-homologous sequence (T7 primer); siRNA, siDNA and siHybrids: 21 base sequence from hamster G6PD.

FIG. 4 illustrates the effects on G6PD expression in both CHO cells and human cells of siRNA (FIG. 4A), siDNA (FIG. 4B), and RNAs:DNAa siHybrid (FIG. 4C). Expression was assayed as in FIG. 2.

Expression level obtained using the positive control was defined as 100%. Key: human cells: white bars; CHO cells: shaded bars; “+”: positive control (no transfection); “−”: negative control (cells incubated with stain not containing G6P); B: blank (transfection without nucleic acid); N: non-homologous sequence (T7 primer); siRNA, siDNA and siHybrids: 21 base sequence homologous to both hamster and human G6PD.

### DETAILED DESCRIPTION

Two of the greatest weaknesses of siRNA are its requirement for aided delivery to cells and its short term effects. Transfection is a strategy to deliver genes and other nucleic acids into eukaryotic cells. There are three categories of transfection techniques: biochemical methods, physical methods and virus mediated methods. The transfection technique used is determined by the stress of the transfection on the cells and the efficiency of the method. Biochemical approaches include calcium-phosphate mediated, DEAE-dextran mediated, and lipotransfection. Physical methods include electroporation and biolistics.

Short duration is a characteristic of siRNA that prevents any meaningful clinical use. Potential applications including cancer therapies, antiviral agents, and cures for certain genetic diseases all require a long-acting process to facilitate delivery and effectiveness.

Disclosed herein are siHybrid molecules that have similar function to siRNA, but are much more effective at gene silencing. Instead of a double-stranded RNA molecule, an siHybrid molecule comprises one strand of nucleic acid, e.g., RNA, hybridized to a second strand of nucleic acid that is a different type of nucleic acid than the first strand, e.g., DNA. The siHybrid created by the hybridization of the two different types of nucleic acid have a hybridized complementary portion and at least one 3′ overhanging end. Nucleic acid analogs can be used in place of nucleic acids. The term “nucleic acid analog” refers to modified or non-naturally occurring nucleotides or backbone structures, such as peptide nucleic acid (PNA).

The unique functions of siHybrids may relate to the stability of the molecule. A double-stranded RNA molecule is inherently unstable; it is rapidly degraded in mammalian cells. A DNA:RNA hybrid, in contrast, is the most stable sort of nucleic acid molecule possible from natural materials, and the construct is not degraded in eukaryotic cells. Experimental results indicate that the DNA:RNA hybrid is a more potent gene silencing agent than siRNA. Logically, the more stable the molecule is, the more potent a gene silencing agent the molecule can be. Therefore, an siHybrid comprising at least one PNA, or a molecule made of new synthetic nucleic acid analogs, might be equally effective or more potent than a DNA:RNA hybrid, if the synthetic siHybrid is more stable than a DNA:RNA hybrid.

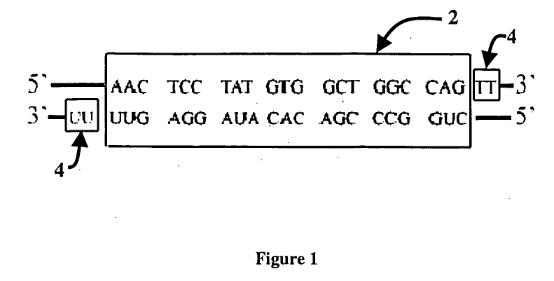

Referring to FIG. 1, the most effective siHybrids have a hybridized complementary portion (2) that is 19 to 21 base pairs in length and at least one overhanging 3′ end (4) that is at least 2 bases in length. The hybridized complementary portion of the molecule can be up to 100 base pairs. Generally, the shorter the length is, the less the specificity there will be. If the siHybrid contains less than ten base pairs, it Will lose specificity for silencing a gene. On the other hand, a long molecule will have difficulty entering a cell, and therefore cannot silence the gene. Thus, an siHybrid containing more than 100 base pairs will have difficulty entering a cell.

An sihybrid with a sequence common to more than one gene can be used to silence multiple genes simultaneously. Also, multiple siHybrids can be used to silence multiple genes. Multiple gene silencing is useful for, e.g., human therapeutic purposes. For example, by suppressing multiple genes responsible for tumor growth, efficient inhibition of the tumor's growth that may not be achieved by suppressing just one gene can be effected.

siHybrid molecules have near universal potential. They can be used to silence genes in the cell(s) of any eukaryotic organism. They can be used for therapy or research purposes. They can be used as antiviral agents and cancer therapy agents and can also be used to treat various genetic diseases caused by the unwanted over-expression of a gene. In addition, they can be used in plants to cure plant diseases, improve plant traits, such as yield, color, environmental tolerance, or quality. By selectively silencing a gene(s), siHybrids can be used as herbicides, insecticides, pesticides and fungicides.

siHybrids can be used to prevent viral infection of cells. By finding the genes that are unique and essential to virus infection, such as proteinase genes or reverse transcriptase genes, constructing corresponding siHybrids and applying those siHybrids to cells, the virus can be killed and viral infection can be cured by silencing the genes.

Furthermore, siHybrids can be used to treat human or animal diseases resulting from over-expression of genes or disease causing genes. Such diseases may include, but are not limited to, autoimmune diseases, tumors, inflammatory disease and hypertension. siHybrids can also be used to suppress normally expressed genes for therapeutic purposes. For example, to enable successful organ transplants, genes relating to immune response for rejection can be suppressed.

Formulation and Routes of Administration:

siHybrids may be formulated in any pharmaceutically acceptable dosage form. For example, the dosage form may be one suitable for intravenous administration in humans. The dosage forms may include pharmaceutically acceptable excipients, carriers, buffers, osmotic agents and the like, which are known in the art. The formulation may include other pharmaceutically active ingredients for combinational therapies. The formulation may also be designed for a specific utility, in a powder, solid, liquid or gaseous form. siHybrids can be administered orally, subcutaneously, intravenously, intracerebrally, intramuscularly, intramedullary, paretemally, transdermally, nasally or rectally. The form the siHybrids are administered depends at least in part on the route by which they are administered.

### EXAMPLES

Experiments were conducted on mammillian cells as outlined in the sections below. The concentration of siHybrid used in mammillian cell experiments ranged from 10 μg per 1×10⁶ cells to 25 μg per 1×10⁶ cells in final concentration. Although these experiments demonstrated that the range was effective in silencing genes, the actual lowest effective concentration could be much lower than 10 μg per 1×10⁶ cells.

Mammalian Cell Summary:

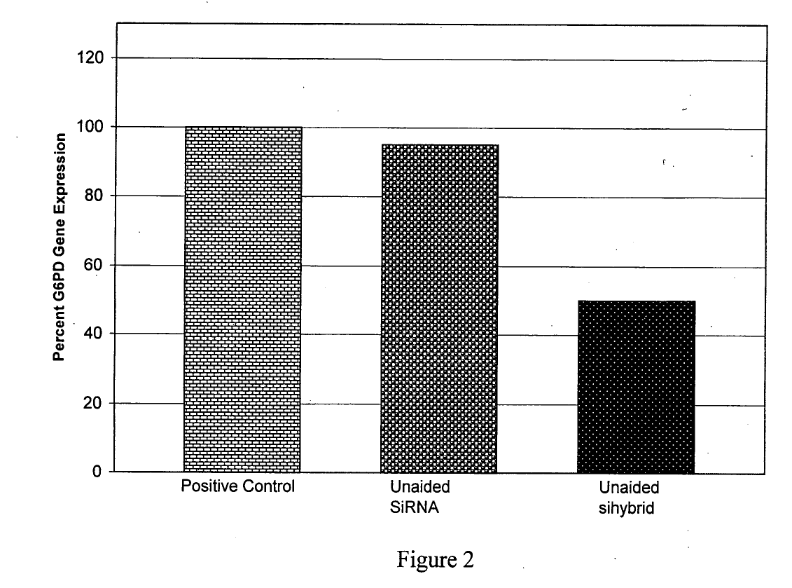

A process was developed to test the effects of siHybrids on various oncogenes and tumor suppressor genes. The goal was to develop a way to shut off a particular gene for a long time, and observe the effects. siHybrids were used to silence the glucose-6-phosphate dehydrogenase (G6PD) gene in normal and cancerous cells of human and hamster origin. The results showed that siHybrids were more potent than siRNA and siDNA in suppressing G6PD gene expression, both in magnitude and duration. The results also showed that the potency of siHybrid is independent of the DNA:RNA orientation. In the siRNA and siHybrid gene silencing experiments only lipotransfection was used. Lipotransfection involves coating the nucleic acid to be delivered into the cells with cationic lipids that bind to the nucleic acid molecules. The artificial membrane fuses with the cell membrane, which is also made of lipids but is negatively charged. For unaided delivery experiments the constructs were added directly to the media. No transfection media or agents were necessary; simply adding the siHybrids to the media was sufficient. FIG. 2 shows gene silencing of G6PD by unaided delivery of siRNA and siHybrid molecules.

In a different experiment, siHybrids were added to dividing cells and then grown for at least eight days. At various intervals during the eight days, attempts were made to induce the G6PD gene, and less than 40% gene expression was observed. Control cells showed normal G6PD activity, and cells in which conventional siRNA molecules had been added showed that normal G6PD activity returned to 100% gene expression within two days. These observations show that sihybrids can be used to silence almost all genes in mammalian cells. This function can be used to suppress any disease causing gene over expression, thus providing an effective treatment for the disease.

Mammalian Cells Experiments

To explore the capabilities of RNAi mediated by siRNA an experiment was designed to post transcriptionally silence an inducible, endogenous gene in cultured mammalian cells, and to determine the duration of this effect. siRNA was used to silence the glucose-6-phosphate dehydrogenase (G6PD) gene in the CHO AA8 cell line, an inducible and endogenous gene found in mammalian cells. G6PD plays an important role in the pentose phosphate pathway in animal tissues to generate the reduced form of nicotinamide dinucleotide triphosphate, NADPH and ribose-5-phosphate that is utilized to generate nucleotides (See Carson, P. E. and Frischer, H. (1966) Glucose-6-Phosphate dehydrogenase deficiency and related disorders of the pentose phosphate pathway. Am J Med. 41, 744-764.). Glucose-6-phosphate enters the pathway and is oxidized by G6PD to generate NADPH and 6-phospho-glucno-δ-lactone (See Carson, P. E. and Frischer, H. (1966) Glucose-6-Phosphate dehydrogenase deficiency and related disorders of the pentose phosphate pathway. Am J Med. 41, 744-764). The oxidative reduction properties of this reaction can be used in combination with a tetrazolium based histochemical stain may be used on cells exposed to Glucose-6-phosphate as a colorimetric assay to quantify the degree of G6PD gene silencing as represented by the level of G6PD enzymatic activity in the cells.

An analysis of siRNA mediated gene silencing with variations in the nucleic acid composition of the short interfering molecules was used to test their effects on the parameters influenced by this mode of gene silencing. These factors include the degree and persistence of the gene silencing effects as well as the amount of recovered gene expression. Using the mammalian G6PD gene these parameters are affected depending on the nucleic acid composition of the short interfering molecules the cells are exposed to. To demonstrate the universality of these findings among mammalian cells a comparison analysis between human and hamster cells was performed.

Materials and Methods

Short interfering molecule preparation. A 21 bp sequence was chosen randomly from the G6PD gene sequence. A second region homologous to a sequence in both the hamster and human G6PD gene was used for the hamster-human comparison studies. Sense and antisense strands were constructed with 2 nucleotide 3′ uridine overhangs at DNA Synthesis Core Facility at Johns Hopkins University. SiDNA sequence contained 2 nucleotide 3′ thymidine overhangs. SiRNA sequence were unpurified, siDNA sequences were RP cartridge purified. Sense and antisense strands were annealed together in equimolar amounts in the presence of 10 mM Tris-HCl (pH 8.0) by denaturing for 5 minutes at 94° C. and reannealed at 53° C. for 3 h and then slowly cooled to room temperature.

Cell Culture and Transfection. Chinese Hamster Ovary (CHO) AA8 cells were propagated in F-12 Nutrient Mixture Ham (Life Technologies, N.Y.) supplemented with 10% Fetal Bovine Serum (FBS), 1% L-glutamine, and 1% antibiotic-antimycotic at 37° C. Human MCF-7 cells were propagated in DMEM/F-12 (Life Technologies) supplemented with 10% FBS, 1% L-glutamine, 1% penicillin-streptomycin, 1% MEM Non essential amino acid solution, 1% sodium pyruvate , and 2% BME amino acid solution. FBS was inactivated by heating for 30 minutes at 56° C. to eliminate nuclease activity. Cells were passed 3 times per week to maintain exponential growth. Twenty four hours prior to transfection cells were washed 3 times with 1×PBS, trypsinized and plated in 35 mm tissue culture dishes at 1×10⁶ cells/plate in 2 ml growth medium without antibiotics and incubated at 37° C. Transfection of short interfering molecules was performed using Lipofectamine Reagent (Life Technologies, N.Y.) according to manufacturer's protocol for adherent cells using 10 μg of nucleic acid. Cells were incubated with transfection complexes for 5 h. To prevent toxicity of the cells, complexes were aspirated and cells were washed 2 times with complete growth medium and incubated at 37° C. in growth medium with antibiotics until ready to assay for G6PD enzymatic activity.

G6PD Colorimetric Assay and Quantification of Enzymatic Activity. G6PD enzymatic activity was monitored as described by Stamato et al, (See Stamato, T. D., Mackenzie, L., Pagani, J. M., and Weinstein, R. 1982) Mutagen treatment of single Chinese Hamster Ovary cells produce colonies mosaic for Glucose-6-phosphate dehydrogenase activity. Somatic Cell Genetics. 8, 643-651). Briefly, monolayers of cells were washed with 2 ml of 0.14 M NaCl/0.012% Triton-X 100 solution and incubated at 37° C. for 1 h in 2 ml of solution containing 2.5 mg/ml glucose-6-phosphate disodium salt, pH 6.5, 0.17 mg/ml phenazine methosulfate, 0.33 mg/ml nitro blue tetrazolium, 0.14 M NaCl, 0.17 mg/ml NADP and 0.012% Triton-X 100. Cells were fixed for 15 min with 2 ml of 10% acetate buffered formalin, washed and dried with nitrogen. To quantify enzymatic activity, average pixel intensities of cells were obtained to represent the degree of color in the cells which is related to the level of G6PD activity. Cells were observed using brightfield light on Zeiss Axiophot at 20x. Images were taken of plates in regions where there were monolayer of cells. The difference in average pixel intensities of individual cells and regions containing no cells to represent background were obtained. Image analyses were performed using Smart Capture VP software.

Dot Blot. CHO AA8 cells transfected with short interfering molecules at time points 0, 6, 12, 18 and 24 hours post transfection were lifted by washing three times with 1×PBS and incubating with trypsin for 5 minutes. Cells were washed with 1×PBS and resuspended in 1×PBS. Cell suspensions (approximately 10⁵ cells) were boiled for 10 minutes at 95° C. to obtain cellular lysates. Hybridization procedures were performed as described in Gibco's Blugene Nonradioactive Nucleic Acid Detection System to detect the presence of the short interfering molecules in the lysates. Probes used were antisense G6PD DNA sequence that had been biotinylated.

Statistics. The values presented in the CHO AA8 siRNA and siDNA time experiments represent the averages of five replicate experiments, the hybrid data represents the averages of three replicate experiments. The values presented of the hamster-human comparison time experiment represents the averages of two replicate experiments. Relative values were obtained by representing the average value of the positive control conditions as 100% and dividing the averages for the experimental conditions by the average positive control value. The error bars represent the standard deviation.

Mammalian Cells Experimental Results

Transfection of short interfering molecules using cationic liposomes inconsistently causes toxicity of cells and yields low transfection efficiency. CHO AA8 and Human MCF-7 cells were transfected with the short interfering molecules using cationic liposomes. Vital counts showed greater than 50% of the cells exposed to the transfection complexes died, regardless of the transfection reagent used. Five different cationic liposome transfection reagents were tried in order to minimize the toxicity and mortality of the cells, with Lipofectamine (Life Technologies) producing the lowest level of cell death. Only cells that looked healthy after transfection were assayed for G6PD activity.

Approximately 40-50% of cells transfected in a 35 mm plate appeared to be transfected with the short interfering molecules, based on cell color when assayed for G6PD enzymatic activity. Transfected and untransfected cells in monolayer cultures tended to occur in discrete patches, as indicated by the color of the cells. Only cells in the transfected regions were analyzed for gene silencing. In control plates where G6PD activity was not inhibited these regions of different intensities of color of the cells were not present, indicating that the G6PD assay was not producing the effect.

A colorimetric assay provided an efficient method to detect the presence of G6PD gene silencing in individual cells. The G6PD gene proved to be an advantageous choice to investigate siRNA-mediated gene silencing. To separate the efficiency of the transfection from the study of siRNA, it was important to be able to assay individual cells rather than obtain a population average. To do this, the enzymatic activity of the G6PD protein was assayed using a colorimetric assay developed by Stamato et al (22). Previous work using siRNA to silence genes in cultured mammalian cells by Elbashir et al (20) also used colorimetric techniques of fluorescent staining and luciferase activity to assay results. After transfection cells were incubated with a tetrazolium-based histochemical stain that contained glucose-6-phosphate (G6P) and nicotinamide dinucleotide triphosphate (NADP). The addition of G6P to cells activated G6PD gene transcription and protein synthesis. The enzymatic activities of G6PD coupled the oxidation of G6P and the reduction of NADP to NADPH, to create a cellular color change from white to purple. If the addition of siRNA with a sequence homologous to the G6PD gene sequence induced post-transcriptional gene silencing in CHO AA8 cells, then an insufficient amount of G6PD protein would be synthesized, resulting in a lack of G6PD enzymatic activity and inhibition of the color change reaction.

A reduction in inducible G6PD enzymatic activity exists in Chinese Hamster cells exposed to siRNA molecules. Relative changes of G6PD activity in siRNA-transfected cells were measured by comparing the color intensity of the cells to non-transfected cells that were also incubated with the histochemical stain. To ensure that the post-transcriptional gene silencing was a specific effect of the siRNA enzymatic activity was also measured in CHO AA8 cells transfected with a non-homologous nucleotide sequence, T7 primer, as well as cells that were exposed to cationic liposomes with no vector. CHO AA8 cells incubated with the histochemical stain in the absence of G6P served as a negative control for the assay. Images of cells were obtained after incubation and the pixel intensities based on the color of individual cells were measured to determine relative changes in G6PD activity.

G6PD activity could be detected in mammalian cells through the coupling of the oxidation of glucose-6-phosphate and the reduction of NADP by G6PD with a tetrazolium based histochemical stain.

Kinetics of siRNA induced gene silencing of G6PD. To determine the kinetics of siRNA post-transcriptional gene silencing of G6PD the colorimetric assay was performed at specific time points over the span of 96 hours after a 5 hour transfection to measure the presence of G6PD enzymatic activity. siRNA mediated gene silencing provided approximately a 60% reduction in G6PD activity for the first 24 hours post transfection. The cells began to regain expression of the G6PD gene at 48 hours and exhibited full expression by 96 hours after transfection.

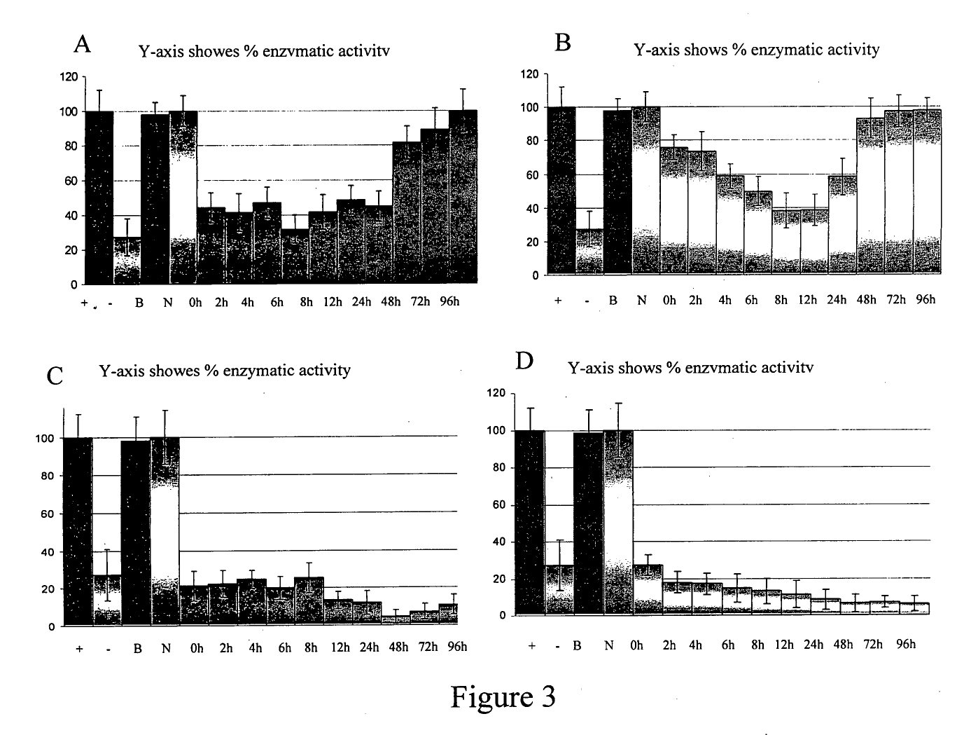

FIG. 3 shows the results of (A) CHO AA8 cells transfected with siRNA molecules, (B) cells exposed to siDNA molecules, (C) introduction of short interfering hybrid molecules DNAs:RNAa, and (D) RNAs:DNAa. Control reactions consisted of transfecting with si molecules (either RNA:RNA, RNA:DNA or DNA:DNA) that had the sequence of the T7 phage promoter primer (T), or exposure to cationic liposome complexes with no vector (B). All cells exposed to control tests exhibited 100% gene expression and enzymatic activity. Cells transfected with siDNA molecules exhibited the lowest degree of gene silencing effects while siRNA molecules provided a greater inhibition of gene expression. The length of silencing lasted approximately 24 hours for cells transfected with siRNA or siDNA molecules. Short interfering hybrid molecules of both DNAs:RNAa and RNAs:DNAa conformations exhibited the greatest degree and persistence of inhibition of endogenous gene expression. Effects continued to persist through 96 hours. Graphs A and B represent data from five replicate experiments and data from graphs C and D represent data from three replicate experiments.

Cells exhibit a differential response in G6PD gene silencing when exposed to short interfering molecules of different nucleic acid composition. Because the mechanism of RNAi mediated by siRNA is not clear it was questioned whether post-transcriptional gene silencing was a specific effect of short interfering sequences made of RNA or could siRNA molecules with variations in their nucleic acid composition provide gene silencing effects. To test this, siDNA sequences and short interfering hybrid molecules composed of both RNA and DNA, identical in sequence to the siRNA vectors used were transfected into CHO AA8 cells and G6PD enzymatic activity was assayed again at designated time points over the span of 96 hours post transfection. Two different hybrid molecules were constructed that differed in which nucleic acid the sense and antisense strands were composed of. Analysis of the cells suggested that a differential response of G6PD silencing existed among the different short interfering molecules used. Cells transfected with siDNA molecules showed the lowest degree of gene silencing and maximum inhibition of expression was not seen until 12 hours post transfection. In contrast cells transfected with siRNA molecules showed a decrease in expression as early as 0 hours after transfection with a greater degree of silencing compared to that provided by the siDNA molecules. Both effects of siDNA and siRNA molecules lasted for approximately 24 hours and normal expression levels were reached by 96 hours.

CHO AA8 cells transfected with the short interfering hybrid molecules of both DNAs:RNAa and RNAs:DNAa exhibited the greatest decrease in G6PD enzymatic activity with the greatest persistence. Cells transfected with DNAs:RNAa showed a decrease in G6PD as early as 0 hours after transfection with percent relative activity at approximately 20%. These effects persisted throughout the time course of the experiment with amount of activity remaining at approximately 20% or lower. Similar effects were seen with cells transfected with RNAs:DNAa molecules. Referring to FIG. 3, percent enzymatic activity remained at or below approximately 20% throughout the experiment. A dot blot was performed to detect the presence of the short interfering hybrid molecules. Nothing was detected, which demonstrates only that the intracellular concentration of the molecules was too low to be detected.

The presence of G6PD activity was assayed for in cells exposed to the hybrid molecules every 24 hours between 120-192 hours post transfection to determine how long the effects last with the hybrid constructs. The presence of G6PD activity increased to about 40% by 120 hours but remained at this level through 192 hours. A dot blot was performed to detect the presence of the short interfering hybrid molecules. Nothing was detected, which demonstrates only that the intracellular concentration of the molecules was too low to be detected.

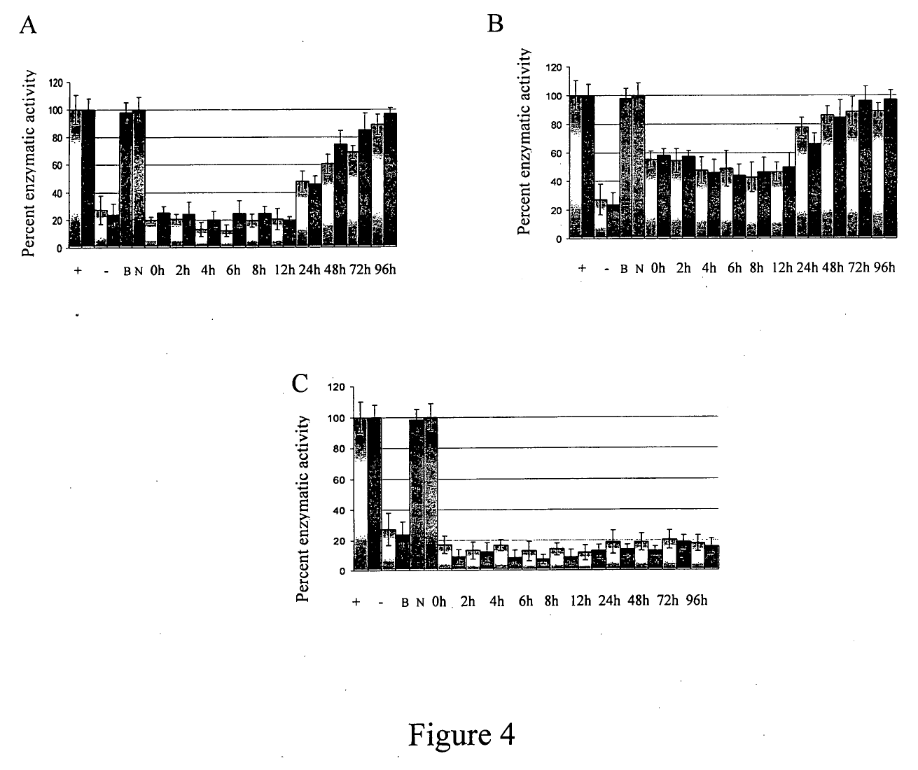

A differential response in siRNA-mediated gene silencing with varied nucleic acid composition possibly exists in all mammalian cells. To show that the differential response was not a specific effect of hamster cells a comparison time course study of the persistence of short interfering molecules with variations in their nucleic acid composition was done in human and hamster cells. This experiment also addressed the effects of varying the sequence of the gene the short interfering molecule is homologous to. The molecules were identical to a sequence in both the hamster and human G6PD coding region. FIG. 4 shows that a differential response was also present in the Human MCF-7 cells suggesting the possible universality of this application to all cultured mammalian cells. Cells transfected with siDNA molecules exhibited the lowest degree of gene silencing while siRNA molecules provided a greater degree of inhibition of gene expression. The silencing effects of both siRNA and siDNA showed a loss by approximately 24 hours post transfection with full expression regained by 96 hours. The hybrid molecules in both human and hamster cells offered the greatest reduction in gene silencing with long term inhibition of endogenous gene expression. Only hybrid molecules composed of a RNA sense strand and a DNA antisense strand were used due to the similarity of the results obtained for both hybrid molecules in the previous experiment involving hamster cells only.

As shown in FIG. 4, CHO AA8 and Human MCF-7 cells were transfected with (A) siRNA molecules (B) siDNA molecules and (C) short interfering hybrid molecule of RNAs:DNAa composition. As the sequence used here is a different sequence then that used in the first series of experiments in the CHO AA8 cells, these results demonstrate both that the differential response was not a cell-specific effect, nor was it a sequence-specific effect. The introduction of siDNA molecules resulted in the lowest inhibition of gene expression, while siRNA molecules provided a greater degree of gene silencing. Both effects in both cell lines lasted for approximately 24 hours post transfection. The short interfering hybrid molecule exhibited the greatest degree and persistence of inhibition of G6PD gene expression that lasted for the time course of the experiment. Data from all three graphs represent data from two replicate experiments.

In addition to showing the potential use this application has in mammalian cells, these experiments demonstrate that a differential response is present regardless of the sequence of the coding region to which the short interfering molecules are homologous. The initial experiments testing the effects of nucleic acid composition in hamster cells utilized a different short interfering sequence than the human-hamster comparison experiment, and both sequences were homologous to undistinguished regions of the coding strand. Yet both resulted in gene silencing with a differential response and a long-term inhibition provided by the hybrid molecules.

All publications and patents mentioned in the above specification are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

The invention claimed is:

1. A method comprising:
providing a single strand of DNA, having 23 bases, wherein 21 bases at the 5′ end are complementary to a target gene;
providing a single strand of RNA, having 23 bases, wherein 21 bases starting at the 5′ end of the single strand of RNA are complementary to 21 bases starting at the 5′ end of the single strand of DNA;
annealing in vitro said single strand of DNA to said single strand of RNA to form an siHybrid having (1) a 21 base pair hybridized portion and (2) a two base over-hanging portion at each 3′ end.

2. The method of claim 1, further comprising:
contacting at least one eukaryotic cell with said siHybrid.

3. A method comprising:
providing a single strand of RNA, having 23 bases, wherein 21 bases starting at the 5′ end are complementary to a target gene;
providing a single strand of DNA, having 23 bases, wherein 21 bases starting at the 5′ end of the single strand of DNA are complementary to 21 bases starting at the 5′ end of the single strand of RNA;
annealing in vitro said single strand of RNA to said single strand of DNA to form an siHybrid having (1) a 21 base pair hybridized portion and (2) a two base over-hanging portion at each 3′ end.

4. The method of claim 3, further comprising:
contacting at least one eukaryotic cell with said sihybrid.

5. A method comprising:
providing a first and a second single strand of nucleic acid, wherein (a) the first strand is DNA and the second strand is RNA or the first strand is RNA and the second strand is DNA and (b) both strands are 23 bases long and (c) the first strand is complementary to a target gene; and annealing in vitro said first single strand and said second single strand to form an sihybrid having (1) a complementary portion 21 bases long and (2) two over-hanging 3′ ends each 2 bases in length.

6. The method of claim 5, further comprising contacting at least one eukaryotic cell with said siHybrid.

7. The method of claims 2, 4, or 6, wherein said eukaryotic cell is a mammalian cell and contacting said cell is performed ex vivo.

8. The method of claims 2, 4, or 6, wherein said eukaryotic cell is a hamster cell or a human cell and contacting said cell is performed ex vivo.

9. The method of claims 1 through 8, wherein the target gene is G6PD.

10. A method comprising:
providing a first and a second single strand of nucleic acid, wherein (a) the first strand is DNA and the second strand is RNA or the first strand is RNA and the second strand is DNA and (b) both strands are 23 bases long and (c) the first strand is complementary to a G6PD gene; annealing in vitro said first single strand and said second single strand to form an siHybrid having (1) a complementary portion 21 bases long and (2) two over-hanging 3′ ends each 2 bases in length; and
contacting a human cell or a hamster cell ex vivo with said siHybrid.

11-18. (canceled)


SEQUENCE LISTING (2 sequences)

SEQ ID NO: 1 - RNA, 23 nt, Artificial Sequence
  Description of Artificial Sequence Synthetic oligonucleotide
cuggcccgacacauaggaguuuu

SEQ ID NO: 2 - DNA, 23 bp, Artificial Sequence
  Description of Artificial Sequence Synthetic oligonucleotide
aactcctatgtggctggccagtt